SEQUENCE LISTING (2 sequences)

SEQ ID NO: 1 - RNA, 19 nt, Artificial Sequence
  si-SMARCA4
cucucucaacgcuguccaa

SEQ ID NO: 2 - RNA, 25 nt, Artificial Sequence
  si-IRAK1
agaacggcuucuacugccuggugua

(19) United States
(12) Patent Application Publication
(10) Pub. No.: US 2023/0272478 A1
(43) Pub. Date: Aug. 31, 2023

(54) BIOMARKER SPECIFIC FOR LIVER CANCER, AND USE THEREOF

(71) Applicant: NEORNAT, Seoul (KR)

(72) Inventors: Suk-Woo NAM, Seoul (KR); Sang-Yean KIM, Seoul (KR)

(73) Assignee: NEORNAT, Seoul (KR)

(21) Appl. No.: 18/006,075

(22) PCT Filed: Jul. 20, 2021
(86) PCT No.: PCT/KR2021/009313

§ 371 Date: Jan. 19, 2023

(30) Foreign Application Priority Data
Jul. 21, 2020 (KR) 10-2020-0090123

(51) Int. Cl. C12Q 1/6886; G01N 33/574

(52) U.S. Cl. CPC C12Q 1/6886; G01N 33/57438; C12Q 2600/158

(57) ABSTRACT

The present invention relates to a use of using, as biomarkers for the detection and diagnosis of hepatocellular carcinoma, genes of which the expression changes specifically to hepatocellular carcinoma, and treating liver cancer by using same as targets. In the present invention, it has been discovered that the expression of SMARCA4 is increased in HCC and SMARCA4 directly increases the expression of IRAK1, and it has been ascertained that oncogenic proteins Gankyrin and AKR1B10 are induced through the transcriptional activation of IRAK1, and thus SMARCA4-IRAK1-Gankyrin and AKR1B10 can be used as markers specific for liver cancer and can be effectively used as targets for liver cancer treatment.

### TECHNICAL FIELD

The present invention relates to a biomarker specific for liver cancer and a use thereof, and more particularly, to a use of using genes of which the expression changes specifically to hepatocellular carcinoma as biomarkers for the detection and diagnosis of hepatocellular carcinoma and treating liver cancer by using same as targets.

### BACKGROUND ART

Cancer is a representative disease that threatens human health and is the most representative cause of death as a single disease in industrialized countries. The cause of cancer is still unknown, but is considered to act with a complex factor of carcinogenic chemicals that act as a genetic factor which is an internal factor, and a cancer-causing factor which is an external factor, continuous inflammation and damage, and cancer-causing viral infections. However, cancer is not hopeless enough to be concluded as an incurable disease, and may be cured with early diagnosis and active treatment. Therefore, early detection, and early diagnosis and treatment are crucial to increasing the effect of cancer treatment, and even in advanced cancer, multifaceted and active methods are employed to cure cancer or prolong the life and improve painful symptoms.

Among cancers, liver cancer is known as one of the most lethal cancers worldwide, and an aggressive cancer with a mortality rate corresponding to the third highest (Ahn J, Flamm S L Hepatocellular carcinoma Dis Mon 2004; 50:556-573). Therapeutic surgery is available for only 15% to 25% of patients, and most liver cancer patients die within a relatively short period of time due to locally advanced or metastatic diseases (Roberts L R, Gores G J Hepatocellular carcinoma: molecular pathways and new therapeutic targets, Semin Liver Dis, 2005; 25:212-225). Hepatitis B virus, hepatitis C virus, aflatoxin B1, and the like are well known as major causes of liver cancer. However, the overall survival rate of liver cancer patients has not increased significantly over the past 20 years, and the mechanisms of development and progression of liver cancer are still unknown well (Bruix J, et al., Focus on hepatocellular carcinoma, Cancer Cell, 2004; 5:215-219). So far, molecular targeted therapy has been effective in the treatment of mature liver cancer (Shen Y C, Hsu C, Cheng A L Molecular targeted therapy for advanced hepatocellular carcinoma: current status and future perspectives, J Gastroenterol; 45:794-807), but it is unclear how these genetic changes cause the clinical characteristics observed in individual patients with liver cancer. The liver cancer may be largely divided into primary liver cancer (hepatocellular carcinoma), which arose from the hepatocytes itself, and metastatic liver cancer, in which cancer of other tissues has metastasized to the liver, but 90% or more of liver cancer is primary liver cancer.

Hepatocellular carcinoma (HCC) is the fifth most common tumor in the world, accounting for 500,000 deaths each year (Okuda 2000). The survival rate of HCC patients has not improved over the past 20 years and has an incidence rate approximately equal to mortality (Marrero, Fontana et al., 2005). Chronic hepatitis caused by infection with hepatitis B virus or hepatitis C virus and exposure to carcinogens such as aflatoxin B1 are known as major risk factors for HCC (Thorgeirsson and Grisham, 2002). It has been reported that changes in cell cycle regulators that progress to the G1 phase in the cell cycle mechanism are involved in the formation of liver cancer (Hui et al., Hepatogasteroenterology 45:1635-1642, 1998), but intracellular molecular mechanisms related to the development and progression of liver cancer have not yet been clearly identified. According to previous studies, when protooncogenes such as various growth factor genes are mutated into oncogenes by various causes and overexpressed or hyperactivated, or when a tumor suppressor gene such as Rb protein or p53 protein is mutated by various causes and underexpressed or lost the function, it has been reported to cause the development and progression of various cancers, including liver cancer. In addition, it has been reported that DNA mutations, genetic alterations in gene expression, and the like are identified in liver cancer patient tissues (Park et al., Cancer Res 59:307-310, 1999; Bjersing et al., J Intern Med, 234:339-340, 1993; Tsopanomichalou et al., Liver 19:305-311, 1999; Kusano et al., Hepatology 29:1858-1862, 1999; Keck et al., Cancer Genet Cytogenet 111:37-44, 1999). Recently, it has been recognized that the development and progression of most cancers, including liver cancer, are not caused by some specific genes, but caused by complex interactions of various genes related to cell cycle, signaling, and the like. Therefore, there is a need for comprehensive research on various genes or proteins, beyond focusing only on the expression or function of individual genes or proteins.

### DISCLOSURE

### Technical Problem

An object of the present invention is to provide a biomarker for diagnosing liver cancer.

Another object of the present invention is to provide a composition for diagnosing liver cancer.

Yet another object of the present invention is to provide a kit for diagnosing liver cancer.

Still another object of the present invention is to provide a pharmaceutical composition for preventing or treating liver cancer.

Still yet another object of the present invention is to provide a method for providing information required for diagnosing liver cancer.

Still yet another object of the present invention is to provide a method for providing information for predicting the prognosis of liver cancer.

Still yet another object of the present invention is to provide a method for predicting the reactivity of a SMARCA4 inhibitor to liver cancer treatment.

### Technical Solution

An aspect of the present invention provides a biomarker for diagnosing liver cancer including one or more genes selected from the group consisting of SMARCA4, SMARCC1, SMARCA2, and IRAK1 or proteins expressed from the genes.

Another aspect of the present invention provides a composition for diagnosing liver cancer, including an agent for measuring the expression level of one or more biomarker genes selected from the group consisting of SMARCA4, SMARCC1, SMARCA2, and IRAK1 at an mRNA or protein level.

Yet another aspect of the present invention provides a kit for diagnosing liver cancer including the composition.

Still another aspect of the present invention provides a pharmaceutical composition for preventing or treating liver cancer including a SMARCA4 inhibitor as an active ingredient.

Still another aspect of the present invention provides a method for providing information required for the diagnosis of liver cancer.

Still another aspect of the present invention provides a method for providing information for predicting the prognosis of liver cancer.

Still yet another aspect of the present invention provides a method for predicting the reactivity of a SMARCA4 inhibitor to liver cancer treatment.

Still yet another aspect of the present invention provides a method for preventing or treating liver cancer including administering, to a subject, a pharmaceutical composition for preventing or treating liver cancer including a SMARCA4 inhibitor as an active ingredient.

### Advantageous Effects

According to the present invention, it was found that the expression of SMARCA4 was increased in HCC, and SMARCA4 directly increased IRAK1 expression, it was confirmed that the oncogenic proteins Gankyrin and AKR1B10 are induced through transcriptional activation of IRAK1. Accordingly, SMARCA4-IRAK1-Gankyrin and AKR1B10 can be used as markers specific for liver cancer, and can be usefully used as targets for liver cancer treatment.

### DESCRIPTION OF DRAWINGS

FIG. 1 is a diagram confirming genetic variation and abnormal expression of SWI/SNF subunit genes in HCC:
A: Mutation rates of SWI/SNF complex subunit genes in HCC;
B: Differential gene expression of SMARCA4, SMARCC1, and SMARCA2 in HCC patients compared with health normal patients (NC) in datasets of TCGA_LIHC, ICGC_LIRI, and Catholic_mLIHC (GSE114654);
C: Differential gene expression of SMARCA4, SMARCC1, and SMARCA2 in HCC tissue and tissue pairs of HCC from noncancerous liver biopsies corresponding thereto;
D: Expression of SMARCA4, SMARCC1, and SMARCA2 in HCC tissue and tissue pairs of HCC from noncancerous liver biopsies corresponding thereto analyzed by qRT-PCR; and
E: Results of Western blot assay of SMARCA4, SMARCC1, and SMARCA2 in 10 pairs of selected HCC subsets.

FIG. 2 is a diagram of analyzing the tumorigenicity of SMARCA4, SMARCC1, and SMARCA2 in HCC:
A: Cell viability of HCC treated with si-SMARCA4;
B: Cell proliferation of HCC treated with si-SMARCA4;
C: Cell viability of HCC treated with si-SMARCC1;
D: Cell proliferation of HCC treated with si-SMARCC1; and
E: Cell viability of SMARCA4 high-expressing cells and SMARCA4 non-expressing cells treated with si-SMARCA2.

FIG. 3 is a diagram of confirming the metastatic potential of SMARCA4 in a mouse HCC model:
A: Motility and invasion of HCC cells treated with si-SMARCA4;
B: Moving cell images;
C: Cell cycle profile of HCC cells treated with si-SMARCA4;
D: Western blot assay results of cell cycle-related proteins in HCC cell lines;
E: Administration schedules, ultrasonographic images, and tumor masses and mass numbers of si-Cont, si-SMARCA4, si-SMARCC1, and pBJ-SMARCA2 in Ras-Tg mouse model; and
F: Western blot assay results of SMARCA4, SMARCC1, and SMARCA2 in liver tissue of Ras-Tg mouse model.

FIG. 4 is a diagram of confirming specific target genes regulated by SMARCA4 in HCC:
A: Pie charts, Venn diagrams, and circuit diagrams of enhancer genes and SMARCA4-regulated genes;
B: Heatmap of 563 SMARCA4-related genes in multistage HCC;
C: Functional assay of 563 genes;
D: Expression assay results of SMARCA4 and IRAK1 in HCC cell lines treated with si-SMARCA4 (qRT-PCR);
E: Results of ChIP-qPCR assay to evaluate SMARCA4 binding to IRAK1 enhancer; and
F: Dual luciferase assay results in HCC cell lines expressing reporter plasmid expressing IRAK1 enhancer region after si-SMARCA4 treatment.

FIG. 5 is a diagram of confirming overexpression of SMARCA4 and IRAK1 in an HCC patient cohort.

FIG. 6 is a diagram of confirming anti-tumor effects in HCC by target-inhibition of IRAK1:
A: Differential gene expression of IRAK1 gene;
B: Cell growth (cell viability) and cell proliferation after knockdown of SMARCA4 or IRAK1 gene;
C: Cell cycle profile;
D: Western blot assay results of cell cycle-related proteins;
E: Cell images and cell numbers identified using Boyden chamber motility assay; and
F: Cell images and cell numbers identified using Transwell invasion assay.

FIG. 7 is a diagram of confirming the regulation of IRAK1 activity of SMARCA4 in HCC:
A: Anti-tumor effect by IRAK1 (pcDNA3.1_IRAK1) after knocking down SMARCA4 (Western blot assay, MTT assay, and BrdU assay);
B: Cell cycle profile;
C: Results of confirming cell cycle regulatory proteins by Western blot assay;
D: Cell images and cell numbers identified using Boyden chamber motility assay; and
E: Cell images and cell numbers identified using Transwell invasion assay.

FIG. 8 is a diagram of confirming that SMARCA4 activates tumor proteins Gankyrin and AKR1B10 by regulating IRAK1 in HCC:
A: Western blot assay results of downstream regulatory molecules of IRAK1;
B: Results of confirming the expression changes of downstream regulatory molecules of IRAK1 by SMARCA4 knockdown (si-SMARCA4) and IRAK1 recovery (pcDNA3.1_IRAK1) by Western blot assay;
C: Administration schedule of Ras-Tg mouse model;
D: Tumor mass numbers;
E: Results of Western blot assay of liver tissue from Ras-Tg mice; and
F: Schematic diagram of mechanism of SMARCA4-IRAK1-Gankyrin and AKR1B10.

### BEST MODE OF THE INVENTION

Hereinafter, an embodiment of the present invention will be described in detail with reference to the accompanying drawings. However, the following embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Terminologies used herein are terminologies used to properly express preferred embodiments of the present invention, which may vary according to a user, an operator's intention, or customs in the art to which the present invention pertains. Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

In the present invention, the term “subject” or “patient” refers to any single individual in need of treatment, including human, ape, monkey, cow, dog, guinea pig, rabbit, chicken, insect, and the like. In addition, any subject participating in clinical research trials that do not show any clinical signs of disease or subjects participating in epidemiological studies or subjects used as a control are included in the subject.

As used herein, the term “sample” refers to a biological sample obtained from a subject or patient. Sources of the biological sample may be fresh, frozen, and/or preserved organ or tissue samples or solid tissue from biopsies or aspirates; blood or any blood component; and cells at any time point of conception or development in a subject.

All technical terms used in the present invention, unless otherwise defined, have the meaning as commonly understood by those skilled in the related art of the present invention. In addition, although preferred methods and samples are described herein, similar or equivalent methods and samples thereto are also included in the scope of the present invention. The contents of all publications disclosed as references in this specification are incorporated in the present invention.

In an aspect, the present invention relates to a biomarker for diagnosing liver cancer including one or more genes selected from the group consisting of SMARCA4 (SWI/SNF related, Matrix associated, actin dependent regulator of chromatin, subfamily a, member 4), SMARCC1, SMARCA2, and IRAK1 (Interleukin-1 receptor-associated kinase 1) or proteins expressed from the genes.

In one embodiment, the biomarker may include genes of SMARCA4 and IRAK1 or proteins expressed from the genes.

In one embodiment, the liver cancer may be hepatocellular carcinoma (HCC), more preferably IRAK1-overexpressing hepatocellular carcinoma.

In one embodiment, the hepatocellular carcinoma may be overexpressed with IRAK1, but is not limited thereto.

In one embodiment, a gene of Gankyrin or AKR1B10 (Aldo-keto reductase family 1 member B10) or a protein expressed from the gene may be further included.

In one embodiment, the expression of SMARCA4, SMARCC1, IRAK1, Gankyrin, or AKR1B10 may be increased specifically for liver cancer, and the expression of SMARCA2 may be decreased specifically for liver cancer.

In one aspect, the present invention relates to a composition for diagnosing liver cancer, including an agent for measuring the expression level of one or more biomarker genes selected from the group consisting of SMARCA4, SMARCC1, SMARCA2, and IRAK1 at an mRNA or protein level.

In one embodiment, the composition may further include an agent for measuring the expression level of Gankyrin or AKR1B10 at the mRNA or protein level.

In one embodiment, the liver cancer may be hepatocellular carcinoma (HCC), more preferably IRAK1-overexpressed hepatocellular carcinoma.

In one embodiment, the composition of the present invention may include an agent for measuring the expression level of the SMARCA4 and IRAK1 genes at the mRNA or protein level.

In one embodiment, the agent for measuring the expression level of the biomarker gene at the mRNA level may include a nucleic acid sequence of the marker, a nucleic acid sequence complementary to the nucleic acid sequence, and a primer pair, a probe, or a primer pair and a probe specifically recognizing fragments of the nucleic acid sequence and the complementary sequence. The measurement may be performed by a method selected from the group consisting of polymerase chain reaction, real-time RT-PCR, reverse transcription polymerase chain reaction, competitive RT-PCR, nuclease protection assay (RNase, Si nuclease assay), in situ hybridization, nucleic acid microarray, Northern blot, or DNA chip.

In one embodiment, the agent for measuring the expression level of the biomarker gene at the protein level may be an antibody, an antibody fragment, an aptamer, an avimer (avidity multimer), or peptidomimetics, that specifically recognizes a full-length protein of the marker or a fragment thereof. The measurement may be performed by a method selected from the group consisting of Western blot, enzyme linked immunosorbent assay (ELISA), radioimmunoassay (RIA), radioimmunodiffusion, immunoelectrophoresis, tissue immunostaining, immunoprecipitation assay, complement fixation assay, FACS, mass spectrometry, or protein microarray.

As used herein, the term “detection” or “measurement” refers to quantifying the concentration of an object to be detected or measured.

In the present invention, the term “primer” refers to a nucleic acid sequence having a short free 3 hydroxyl group, and a short nucleic acid sequence capable of forming a base pair with a complementary template and serving as a starting point for copying a template strand. The primer may initiate DNA synthesis in the presence of a reagent for polymerization (i.e., DNA polymerase or reverse transcriptase) and four different nucleoside triphosphates in appropriate buffer and temperature.

In the present invention, the term “probe” refers to a nucleic acid fragment such as RNA or DNA corresponding to several bases to several hundred bases in length capable of forming a specific binding to mRNA, and is labeled to identify the presence or absence of a specific mRNA. The probe may be prepared in the form of an oligonucleotide probe, a single stranded DNA probe, a double stranded DNA probe, an RNA probe, or the like. Selection of an appropriate probe and hybridization conditions may be modified based on those known in the art, and thus, the present invention is not particularly limited thereto.

The primer or probe of the present invention may be chemically synthesized using a phosphoramidite solid support method, or other well-known methods. Such a nucleic acid sequence may also be modified using many means known in the art. Non-limiting examples of such a modification include methylation, encapsulation, substitution with one or more homologs of natural nucleotides, and modifications between nucleotides, for example, modification to uncharged linkers (e.g., methyl phosphonate, phosphotrieste phosphoroamidate, carbamate, etc.) or charged linkers (e.g., phosphorothioate, phosphorodithioate, etc.).

In the present invention, suitable conditions for hybridizing the probe with a cDNA molecule may be determined in a series of processes by an optimization procedure. This procedure is performed as a series of processes by those skilled in the art to establish a protocol for use in a laboratory. For example, conditions such as temperature, concentration of components, hybridization and washing time, buffer components, and pH and ionic intensities thereof depend on various factors such as a length and a GC amount of the probe, a target nucleotide sequence, and the like. Detailed conditions for hybridization may be identified in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). For example, under the stringent conditions, a high stringent condition means hybridization at a condition of 65° C. in 0.5 M NaHPO₄, 7% sodium dodecyl sulfate (SDS), and 1 mM EDTA, and washing at a condition of 68° C. in 0.1×SSC (Standard saline citrate)/0.1% SDS. Alternatively, the high stringent condition means washing at a condition of 48° C. in 6×SSC/0.05% sodium pyrophosphate. A low stringent condition means washing at a condition of 42° C. in 0.2×SSC/0.1% SDS.

As used herein, the term “antibody” is a term known in the art and refers to a specific protein molecule directed against an antigenic site. The antibody includes a partial peptide that may be made from the protein. The form of the antibody of the present invention is not particularly limited, and polyclonal antibodies, monoclonal antibodies, or some antibodies having antigen-binding properties are also included in the antibody of the present invention, and all immunoglobulin antibodies are included. Furthermore, the antibody of the present invention includes special antibodies such as humanized antibodies.

In one aspect, the present invention relates to a kit for diagnosing liver cancer including the composition of the present invention.

In one embodiment, the kit may further include not only tools and/or reagents for collecting a biological sample from a subject or patient, but also tools and/or reagents for preparing genomic DNA, cDNA, RNA, or protein from the sample. For example, the kit may include a PCR primer for amplifying a relevant region of genomic DNA. The kit may include a probe of a genetic factor useful for pharmacogenomic profiling. In addition, in the use of such a kit, labeled oligonucleotides may be used for easy identification during analysis.

As used herein, the term ‘diagnosis’ includes determining the susceptibility of a subject to a specific disease or disorder, determining whether a subject currently has a specific disease or disorder, determining the prognosis of a subject suffering from a specific disease or disorder (e.g., identifying a pre-metastatic or metastatic cancer state, determining a stage of cancer, or determining the responsiveness of cancer to treatment), or therametrics (e.g., monitoring the condition of a subject to provide information about treatment efficacy).

In one embodiment, the kit may further contain DNA polymerase and dNTP (dGTP, dCTP, dATP, and dTTP), a labeling material such as a fluorescent substance.

In one aspect, the present invention relates to a pharmaceutical composition for preventing or treating liver cancer including a SMARCA4 inhibitor as an active ingredient.

In one embodiment, the SMARCA4 inhibitor may be siRNA, and may be siRNA represented by SEQ ID NO: 1.

In one embodiment, the SMARCA4 inhibitor may further include SMARCA2 or an expression promoter thereof, a Gankyrin inhibitor, an AKR1B10 inhibitor, a SMARCC1 inhibitor, or an IRAK1 inhibitor.

In one embodiment, the IRAK1 inhibitor may be siRNA, and may be siRNA represented by SEQ ID NO: 2.

In one embodiment, the liver cancer may be hepatocellular carcinoma (HCC), more preferably IRAK1-overexpressed hepatocellular carcinoma.

The pharmaceutical composition according to the present invention may include all materials capable of inhibiting the expression of SMARCA4, SMARCC1, or IRAK1 or promoting the expression of SMARCA2.

The pharmaceutical composition of the present invention may further include a pharmaceutically acceptable carrier. The “pharmaceutically acceptable” refers to a composition that is physiologically acceptable and does not cause an allergic reaction, such as gastrointestinal disorder, dizziness, etc., or a similar reaction thereto when administered to humans. The pharmaceutically acceptable carrier includes, for example, carriers for oral administration such as lactose, starch, cellulose derivatives, magnesium stearate, and stearic acid, and carriers for parenteral administration such as water, suitable oil, saline, aqueous glucose, and glycol, etc., and may further include a stabilizer and a preservative. A suitable stabilizer includes antioxidants such as sodium hydrogen sulfite, sodium sulfite, or ascorbic acid. A suitable preservative includes benzalkonium chloride, methyl- or propyl-paraben and chlorobutanol. Other pharmaceutically acceptable carriers may refer to carriers disclosed in the following document (Remington's Pharmaceutical Sciences, 19th ed., Mack Publishing Company, Easton, Pa., 1995).

The pharmaceutical composition according to the present invention may be formulated in a suitable form according to a method known in the art together with the pharmaceutically acceptable carrier as described above. That is, the pharmaceutical composition of the present invention may be prepared in various forms for parenteral or oral administration according to known methods, and a representative formulation for parenteral administration is preferably an isotonic aqueous solution or suspension for injection formulation. The injection formulation may be prepared according to techniques known in the art using a suitable dispersing or wetting agent and a suspending agent. For example, the injection formulation may be formulated for injection by dissolving each component in saline or buffer. In addition, the formulation for oral administration is not limited thereto, but includes powders, granules, tablets, pills, capsules, and the like. The pharmaceutical composition formulated by the method described above may be administered in an effective amount through various routes including oral, transdermal, subcutaneous, intravenous, or intramuscular routes, and the “administration” means introducing a predetermined material into a patient by any appropriate method, and the route of administration of the material may be administered through any general route as long as the material may reach the target tissue. Here, the “effective amount” refers to an amount that exhibits a preventive or therapeutic effect when administered to a patient. The dosage of the pharmaceutical composition according to the present invention may vary depending on various factors such as the type and severity of a disease, age, sex, weight, sensitivity to drugs, a type of current treatment, an administration method, a target cell, etc. of a patient and may be easily determined by experts in the art. In addition, the pharmaceutical composition of the present invention may be administered in combination with conventional therapeutic agents, and may be administered sequentially or simultaneously with conventional therapeutic agents, and may be administered singly or multiply. Preferably, an amount capable of obtaining the maximum effect may be administered with a minimum amount without side effects in consideration of all the factors, and an effective dose of more preferably 1 to 10000 μg/weight kg/day, much more preferably 10 to 1000 mg/weight kg/day may be repeatedly administered several times a day.

In one embodiment, a SMARCA2 expression promoter may include, preferably, a chemical material, a nucleotide, a vector containing the gene, a protein in which the gene is translated or a fragment thereof, or a natural product extract as an active ingredient.

As used herein, the term “expression promotion” means causing the expression of a target gene mRNA or enhancement of translation to a protein.

In one embodiment, an expression inhibitor of SMARCA4, SMARCC1, IRAK1, Gankyrin, or AKR1B10 may include, preferably a chemical material, a nucleotide, an antisense, an siRNA oligonucleotide, or a natural product extract as an active ingredient, more preferably an antisense or small interference RNA (siRNA) oligonucleotide having a sequence complementary to a nucleotide sequence of the gene as an active ingredient.

As used herein, the term “expression inhibition” means causing a decrease in expression of a target gene mRNA or translation to a protein, and preferably means that the expression of the target gene becomes undetectable or exists at an insignificant level thereby.

As used herein, the term “siRNA (Small interfering RNA)” refers to a nucleic acid molecule capable of mediating RNA interference or gene silencing (see International Patent Publication Nos. 00/44895, 01/36646, 99/32619, 01/29058, 99/07409, and 00/44914). Since siRNA may suppress the expression of the target gene, the siRNA is provided as an efficient gene knockdown method or a gene therapy method. The siRNA molecule of the present invention may have a structure forming a double chain in which a sense strand (sequence corresponding to the mRNA sequence of SMARCA4, SMARCC1, IRAK1, Gankyrin, or AKR1B10) and an antisense strand (sequence complementary to the mRNA sequence of SMARCA4, SMARCC1, IRAK1, Gankyrin, or AKR1B10) are located opposite each other, and the siRNA molecule of the present invention may also have a single-stranded structure with self-complementary sense and antisense strands. Furthermore, siRNA may include a part which is not paired by mismatch (corresponding bases are not complementary), bulge (there is no corresponding base on one chain), etc. without limiting that a double-stranded RNA part that pairs RNAs with each other is completely paired. In addition, when a siRNA end structure may suppress the expression of the SMARCA4, SMARCC1, IRAK1, Gankyrin, or AKR1B10 gene by an RNAi effect, either a blunt end or a cohesive end is possible, and a cohesive end structure can be both a 3′-end protruding structure and a 5′-end protruding structure. In addition, the siRNA molecule of the present invention may have a form in which a short nucleotide sequence is inserted between the self-complementary sense and antisense strands, and in this case, the siRNA molecule formed by the expression of the nucleotide sequence forms a hairpin structure by intramolecular hybridization and, as a whole, forms a stem-and-loop structure (shRNA). This stem-and-loop structure is processed in vitro or in vivo to generate active siRNA molecules capable of mediating RNAi. Methods for producing siRNA include a method of directly synthesizing siRNA in vitro and then introducing siRNA into cells through a transformation process, and a method of transforming or infecting, into cells, siRNA expression vectors or PCR-derived siRNA expression cassettes prepared so that siRNA is expressed in cells.

In one embodiment, the composition of the present invention including gene-specific siRNA may include an agent that promotes the endocytosis of siRNA. The agent that promotes the endocytosis of siRNA may generally use agents that promote nucleic acid introduction. For example, liposomes may be used or one lipophilic carrier of many sterols, including cholesterol, cholate, and deoxycholic acid, may be mixed. In addition, cationic polymers such as poly-L-lysine, spermine, polysilazane, polyethylenimine (PEI), polydihydroimidazolenium, polyallylamine, and chitosan may be used, and anionic polymers such as succinylated PLL, succinylated PEI, polyglutamic acid, polyaspartic acid, polyacrylic acid, polymethacylic acid, dextran sulfate, heparin, and hyaluronic acid may be used.

In one embodiment, when an antibody specific to the protein is used as a material that reduces the expression and activity of the SMARCA4, SMARCC1, IRAK1, Gankyrin, or AKR1B10 protein, the antibody may be directly or indirectly through a linker or the like coupled (e.g., covalently bound) with an existing therapeutic agent.

As used herein, the term “antisense oligonucleotide” refers to DNA or RNA or derivatives thereof containing a nucleic acid sequence complementary to a specific mRNA sequence, and serves to inhibit the translation to the SMARCA4, SMARCC1, IRAK1, Gankyrin, or AKR1B10 protein by binding to a complementary sequence in mRNA. The antisense sequence of the present invention refers to a DNA or RNA sequence that is complementary to SMARCA4, SMARCC1, IRAK1, Gankyrin, or AKR1B10 mRNA and may bind to SMARCA4, SMARCC1, IRAK1, Gankyrin, or AKR1B10 mRNA, and may inhibit the essential activity for translation, translocation into the cytoplasm, maturation, or other overall biological functions of SMARCA4, SMARCC1, IRAK1, Gankyrin, or AKR1B10 mRNA. In addition, the antisense nucleic acid may be modified at one or more bases, sugars or backbone positions to enhance efficacy (De Mesmaeker et al., Curr Opin Struct Biol., 5, 3, 343-55, 1995). The nucleic acid backbone may be modified with phosphorothioate, phosphotriester, methyl phosphonate, short-chain alkyl, cycloalkyl, short-chain heteroatomic, heterocyclic intersugar linkages, and the like. In addition, the antisense nucleic acid may contain one or more substituted sugar moieties. The antisense nucleic acid may contain modified bases. The modified bases include hypoxanthine, 6-methyladenine, 5-methylpyrimidine (particularly, 5-methylcytosine), 5-hydroxymethylcytosine (HMC), glycosyl HMC, gentiobiosyl HMC, 2-aminoadenine, 2-thio uracil, 2-thiothymine, 5-bromouracil, 5-hydroxymethyluracil, 8-azaguanine, 7-deazaguanine, N6(6-aminohexyl)adenine, 2,6-diaminopurine, and the like. In addition, the antisense nucleic acid of the present invention may be chemically bound with one or more moieties or conjugates that enhance the activity and cell adhesion of the antisense nucleic acid. The antisense nucleic acid includes fat-soluble moieties such as cholesterol moiety, cholesteryl moiety, cholic acid, thioether, thiocholesterol, fatty chain, phospholipid, polyamine, polyethylene glycol chain, adamantane acetic acid, palmityl moiety, octadecylamine, hexylaminocarbonyl-oxycolesterol moieties, and the like, but is not limited thereto. Oligonucleotides containing fat-soluble moieties and preparation methods are already well known in the art of the present invention (U.S. Pat. Nos. 5,138,045, 5,218,105, and 5,459,255). The modified nucleic acid may increase the stability to nucleases and increase binding affinity between the antisense nucleic acid and the target mRNA. The antisense oligonucleotide may be synthesized in vitro by a conventional method to be administered in vivo, or may be synthesized in vivo. One example of synthesizing the antisense oligonucleotide in vitro is using RNA polymerase I. One example of synthesizing the antisense RNA in vivo is to allow the antisense RNA to be transcribed by using a vector having the origin of a multicloning site (MCS) in an opposite direction. The antisense RNA is preferably not translated into a peptide sequence so that a translation stop codon is present in the sequence.

In an aspect, the present invention relates to a method for providing information required for the diagnosis of liver cancer, including (a) measuring a gene expression level of SMARCA4 or IRAK1 in a biological sample isolated from a test subject; (b) comparing a result of the corresponding gene of a normal control sample; and (c) determining that the test subject has liver cancer when the gene expression level in step (a) is higher than the expression level of the corresponding gene in step (b).

In an aspect, the method may further include (a) measuring a gene or protein expression level of SMARCC1, SMARCA2, Gankyrin, or AKR1B10 in a biological sample isolated from a test subject; (b) comparing a result of the corresponding gene or protein of a normal control sample; and (c) determining that the test subject has liver cancer, when the expression level of SMARCC1, Gankyrin, or AKR1B10 in step (a) is higher than the expression level of the corresponding gene or protein in step (b), or the expression level of SMARCA2 in step (a) is lower than the expression level of the corresponding gene or protein in step (b).

In one embodiment, the biological sample may include samples such as tissue, cells, whole blood, serum, plasma, saliva, sputum, cerebrospinal fluid, or urine.

In an aspect, the present invention relates to a method for providing information for predicting the prognosis of liver cancer, including (a) measuring a gene expression level of SMARCA4 or IRAK1 in a biological sample isolated from a test subject; (b) comparing a result of the corresponding gene of a normal control sample; and (c) determining that the prognosis of the test subject is poor when the gene expression level in step (a) is higher than the expression level of the corresponding gene in step (b).

In one aspect, the present invention relates to a method for predicting responsiveness of a SMARCA4 inhibitor to liver cancer treatment, including measuring the expression level of IRAK1.

In one embodiment, the method may further include measuring the expression level of Gankyrin or AKR1B10.

In one aspect, the present invention relates to a method for preventing or treating liver cancer including administering, to a subject, a pharmaceutical composition for preventing or treating liver cancer including a SMARCA4 inhibitor as an active ingredient.

### Modes of the Invention

Hereinafter, the present invention will be described in more detail through Examples. However, these Examples are more specifically illustrative of the present invention, and the scope of the present invention is not limited to these Examples.

### Example 1. Evaluation of Expression Levels of Subunit Genes of SWI/SNF Complex in HCC

To confirm the expression levels of subunit genes in HCC, data was obtained from a Gene Expression Omnibus (GEO) database (Accession Numbers: GSE6764, GSE22058, GSE25097, GSE54238, GSE62043, GSE77314, GSE89377, and GSE114564) of TCGA_LIHC (The Cancer Genome Atlas liver hepatocellular carcinoma project), ICGC_LIRI (International Cancer Genome Consortium liver Cancer-RIKEN, JP), and NCBI. Level 3 mRNA expression data of TCGA-LIHC HTSeq-FPKM was used to transform log 2 and evaluate a [log 2(fpkm+1)] gene expression level. As a result, human HCC (cBioPortal; n=1,019) showed a very low mutation rate of an SWI/SNF subunit gene (FIG. 1A). The low incidence and heterogeneity of such a genetic alteration suggest that the dominant aberrant expression of a specific subunit gene may cause systemic disturbances in an SWI/SNF complex. Accordingly, as a result of examining the expression of the SWI/SNF subunit gene using HCC data (Catholic_mLIHC; GSE114564), TCGA_LIHC, and ICGC_LIRI sets (FIG. 1B), among 10 SWI/SNF subunit genes, SMARCA4 and SMARCC1 were significantly overexpressed in HCC patients, while SMARCA2 was down-regulated (≥±1.5 fold, P<0.05). In addition, in HCC data (TCGA_LIHC, ICGC_LIRI, GSE77314) containing the same patient information, it was confirmed that SMARCA4 and SMARCC1 were overexpressed in non-cancerous liver tissue and HCC tissue of the same patient, and SMARCA2 was down-regulated at the same time (≥±1.5 fold, P<0.05) (FIG. 1C). In addition, 34 HCC tissues and HCC tissue pairs of noncancerous liver biopsies corresponding thereto were obtained from the National Biobank in Korea and analyzed by qRT-PCR. As a result, abnormal regulation of SMARCA4, SMARCA2, and SMARCC1 was identified even in a tissue pair of matched HCC in TCGA_LIHC, ICGC_LIRI, and GSE77314 (FIG. 1C). In addition, as a result of additional validation by qRT-PCR in an additional set of 34 randomly selected HCC tissues, overexpression of SMARCA4 (67.6%) and SMARCC1 (52.9%) and inhibition of SMARCA2 (61.8%) were consistently observed in this set (FIG. 1D). In addition, as a result of analyzing 10 randomly selected tissue pairs by Western blot, significant overexpression of SMARCA4 and SMARCC1 and inhibition of SMARCA2 were simultaneously observed in HCC (FIG. 1E).

### Example 2. Antitumor Effect by Regulating Expression of SMARCA4, SMARCA2, and SMARCC1

2-1. In Vitro Analysis

In Example above, in order to confirm which role of SMARCA4, SMARCA2, and SMARCC1, of which expression was specifically changed in HCC, was played in hepatocarcinogenesis, each gene was knocked down by RNA interference. To this end, in liver cell lines Hep3B, Huh7, and SNU-449 in which the expression of SMARCA4 and SMARCC1 was relatively higher than that of normal hepatocytes and the expression of SMARCA2 was relatively lower than that of normal hepatocytes, each gene was knocked-down by RNA interference, and cell viability and cell proliferation were confirmed by MTT assay, BrdU assay, and Clonogenic assay. In addition, migration and invasion of knocked-down HCC cells of each gene were confirmed by Boyden chamber motility assay and wound healing assay. In addition, an effect of knockdown of each gene on cell cycle regulation in HCC cells was confirmed by flow cytometric assay, and cell cycle regulatory proteins were confirmed by Western blot assay.

As a result, it was found that the knockdown of SMARCA4 and SMARCC1 significantly suppressed the cell growth and proliferation rate of HCC cells (FIGS. 2A to 2D). In addition, in both SMARCA4-null HCC cell lines, PLC/PRF/5 and SK-Hep-1, the knockdown of SMARCA2 exhibited the overall lethality of HCC cells, but Hep3B cells and Huh7 cells highly expressing SMARCA4 showed no change in growth rate (FIG. 2E). These results indicate a pivotal role of the SMARCA4-SWI/SNF complex in hepatocellular carcinogenesis. In addition, in the result of the Boyden chamber motility assay, it was shown that SMARCA4 knockdown significantly inhibited the serum-stimulated migration and invasion responses of HCC cells (FIG. 3A), and even in the result of the wound healing assay, similarly thereto, it was found that the SMARCA4 knockdown reduced the wound healing effect of HCC cells (FIG. 3B). In addition, as a result of confirming an effect of SMARCA4 knockdown on cell cycle regulation of HCC cells by flow cytometry, it was found that the SMARCA4 knockdown increased the number of cells in a G1 phase in HCC cells (FIG. 3C). As a result of confirming the expression of cell cycle regulatory proteins by Western blot, the SMARCA4 knockdown selectively induced the expression of p27 Kip1 in HCC cells, and simultaneously inhibited cyclin D1, cyclin E, cyclin-dependent kinase 2 (CDK2), CDK4, and CDK6 to induce pRb hypophosphorylation (p-pRb) (FIG. 3D). Through this, it could be seen that the hyperactivity of SMARCA4 in HCC promoted the transition from G1 phase to S phase by regulating electrons of the cell cycle proteins.

2-2. In Vivo Analysis

To determine whether regulation of SMARCA4, SMARCA2, and SMARCC1 induced a tumor inhibitory effect in vivo, 8 Ras-Tg (H-ras12V homozygous transgenic) male mice developed with HCC at about 15 to 18 weeks of age were intravenously injected with a liver-specific delivery reagent Invivofectamine mixed with SMARCA4-targeting mouse siRNA (si-SMARCA4) or SMARCC1 siRNA (si-SMARCC1) from 14 weeks of age or Turbofect mixed with a mouse SMARCA2 expression plasmid (pBJ-SMARCA2) (FIG. 3E). Liver HCC in mice was confirmed by ultrasonography (Philips, Amsterdam, Nederland) from 14 weeks of age to 24 weeks of age when the liver was obtained, the obtained liver weight and tumor mass number were confirmed and crushed, and the HCC was homogenized to confirm the expression of SMARCA4, SMARCA2 and SMARCC1 by western blot assay.

As a result, in a control mouse (si-Cont), HCC was detected from 18 weeks of age, and 3 of 4 mice developed a large number of large HCC. On the other hand, no HCC was detected in the si-SMARCA4-treated mice until 24 weeks of age. However, HCC was detected at 20 weeks of age in mice of a si-SMARCC1-treated group and mice of a pBJ-SMARCA2-treated group, and relatively small HCC was developed in 3 of 4 mice. Total liver weight change and tumor mass number indicated that target-inhibition of SMARCA4 was remarkably effective in reducing tumor load. In addition, as a result of Western blot assay, inhibition of SMARCA4 and SMARCC1 and increased expression of SMARCA2 were confirmed in the mouse liver tissue of each group (FIG. 3F).

### Example 3. Identification of SMARCA4-Regulated Genes in HCC

3-1. Screening of SMARCA4-Regulated Genes

To identify SMARCA4-SWI/SNF-regulated genes, positions enriched in H3K27ac and H3K27me3, markers specifically recognized by SMARCA4 in HCC cells, were comprehensively mapped using histone ChIP-seq of HepG2 cells available from Encyclopedia of DNA Elements (ENCODE) (https://www.encodeproject.org/). Here, trimmed read values were aligned to a human genome using a Bowtie 2 (http://bowtie-bio.sourceforge.net/bowtie2/index.shtml) mapper, and in order to remove PCR bias, redundant read values were removed using a Sambamba tool (https://lomereiter.github.io/sambamba/). Positions enriched in H3K27ac or H3K27me3 were identified using HOMER (FindPeaks) (FDR-adjusted p-value cutoff=0.001). Through the analysis, 9,198 genes of 21,684 genes closest to the peak were identified as active enhancers (FIG. 4A). Thereafter, in order to identify regulated genetic elements in HCC cells, SMARCA4 knockdown was performed in Hep3B to find 1,865 genetic elements regulated by SMARCA4 (>±1.5 fold, P<0.05), and among them, 1,054 genes were found to be down-regulated in HCC cells in which SMARCA4 was knocked down. As a result of analyzing these 1,054 SMARCA4-regulated genes together with 9,198 active enhancers by Venn diagram analysis, 563 genes were analyzed to be regulated by SMARCA4-SWI/SNF in HCC (FIG. 4A). 563 SMARCA4-related genes showed gradual increases or decreases in transcript levels through different stages of HCC, and showed consistently highest or lowest levels in advanced HCC (FIG. 4B). Using Gene Ontology terms, genes related to carcinogenesis functions including angiogenesis, Wnt signaling, integrin signaling, PDGF signaling, and G-protein signaling were searched in the 563 gene sets (FIG. 4C). In order to select genes regulated by SMARCA4 in HCC, 563 genes were reduced to 84 genes overexpressed in both TCGA_LIHC and ICGC_LIRI datasets 1.5 fold, P<0.05), and thereafter, among 37 genes significantly correlated with the SMARCA4 expression, the top 5 up-regulated genes were examined and validated (FIG. 4A). To confirm whether SMARCA4 directly regulated these genes, HCC cells were knocked down with SMARCA4 siRNA (si-SMARCA4) and gene expression changes were confirmed by qRT-PCR. As a result, it was confirmed that IRAK1 was consistently inhibited by SMARCA4 knockdown in all HCC cells (FIG. 4D).

3-2. Confirmation of Interaction of SMARCA4 and IRAK1

In order to confirm whether SMARCA4 specifically bound to an enhancer region of two genes IRAK1 selected in Example above, ChIP analysis was performed using anti-SMARCA4 and anti-H3K27ac antibodies, respectively, and enrichment rates in an active enhancer region (H3K27ac) of IRAK1 were confirmed. Chromatin immunoprecipitation (ChIP) assay was performed according to the instructions of a manufacturer (Pierce Agarose ChIP kit; Thermo Fisher Scientific, Waltham, Mass.), and DNA was amplified by RT-qPCR using a primer for an enhancer region of each candidate gene regulated by SMARCA4. Cross-linked DNA was precipitated using SMARCA4 antibody (anti-SMARCA4) and H3K27ac (Acetylated histone H3 at lysine 27) antibody (Abcam, Cambridge, UK) (anti-H3K27ac). The experiment was repeated three times and normalized to IgG to obtain an average. In addition, in order to confirm whether endogenous SMARCA4 bound directly to the IRAK1 enhancer region, after cloning the IRAK1 enhancer into a pGL4.23 reporter vector, a dual luciferase reporter assay of a pGL4.23_IRAK1 enhancer was performed in the presence or absence of SMARCA4 in HCC cells.

As a result, in all HCC cells, the enrichment rate of the IRAK1 active enhancer region was significantly reduced by SMARCA4 knockdown compared to a negative control (randomly selected non-SMARCA4-regulated genes GNLS, EIF4G2, and CCT2), but in the same cells, a CKAP4 active enhancer region was consistently not affected by SMARCA4 knockdown (FIG. 4E). In addition, as a result of the luciferase reporter assay, SMARCA4 knockdown significantly inhibited the relative luciferase activity in all HCC cells, so that the interaction between SMARCA4 and the IRAK1 enhancer was confirmed (FIG. 4F).

### Example 4. Confirmation of Overexpression of SMARCA4 and IRAK1 in HCC Patient Cohort

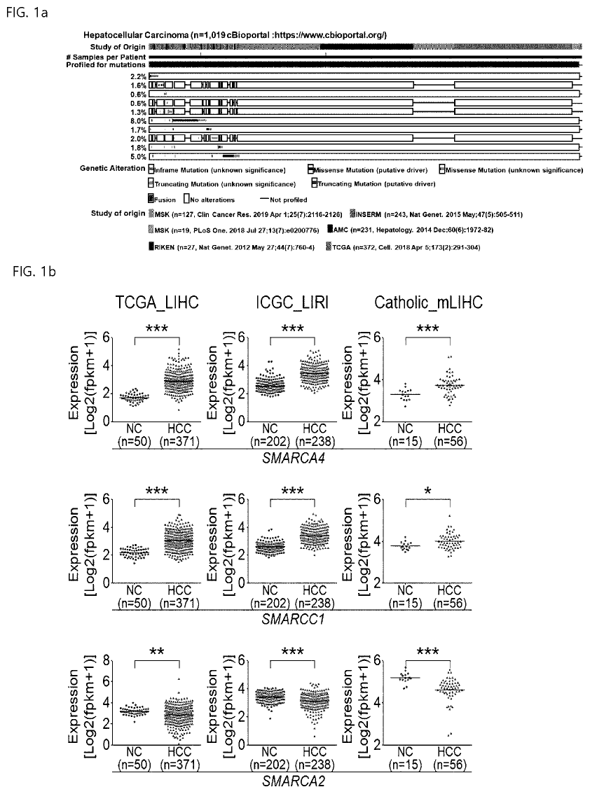

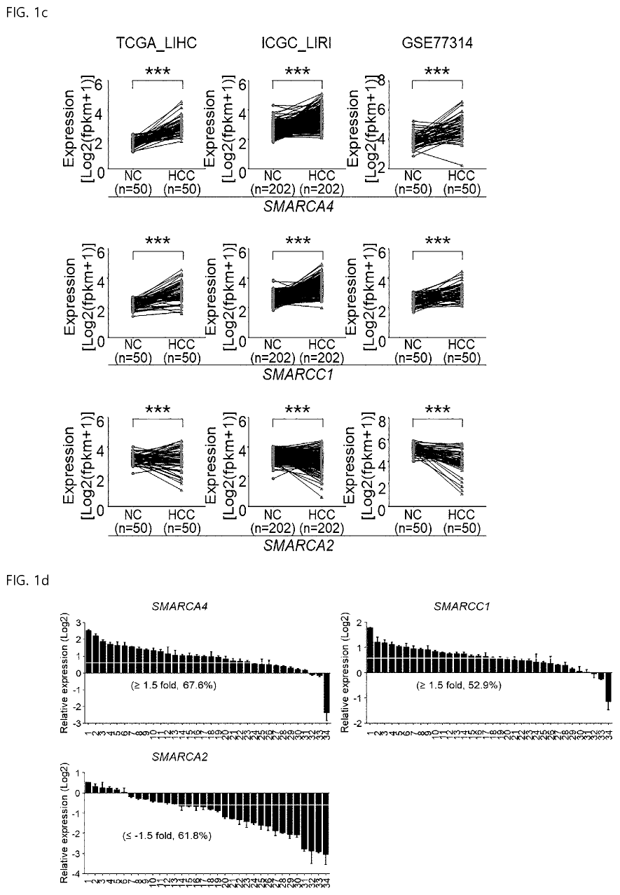

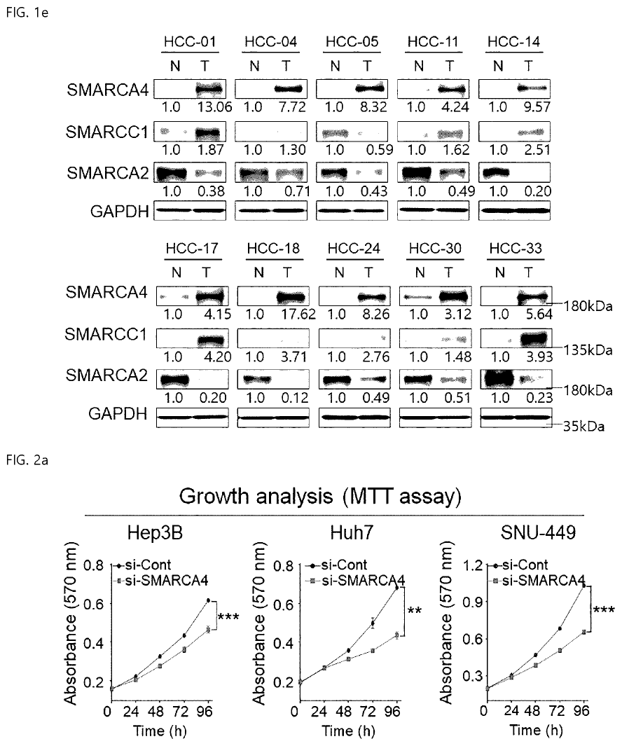

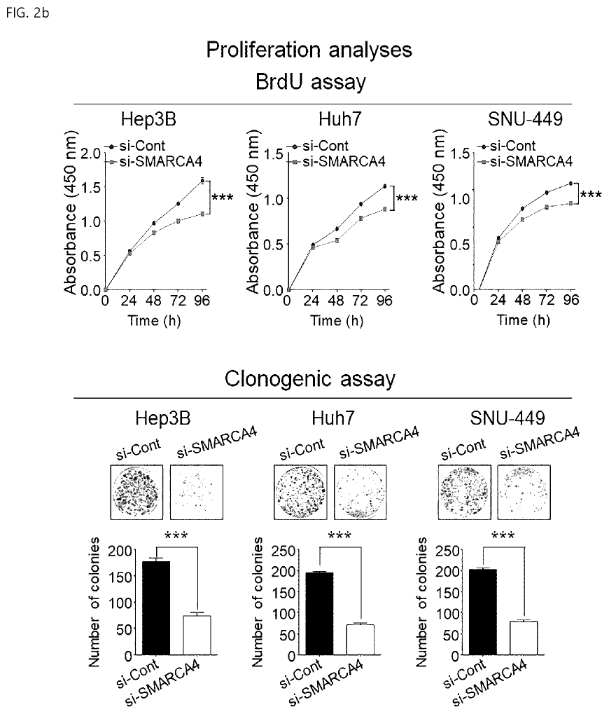

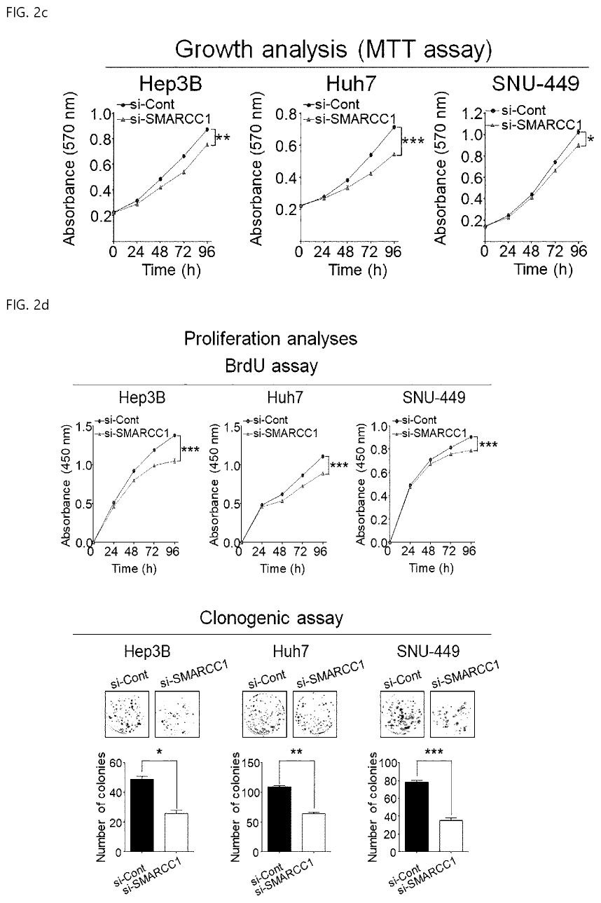

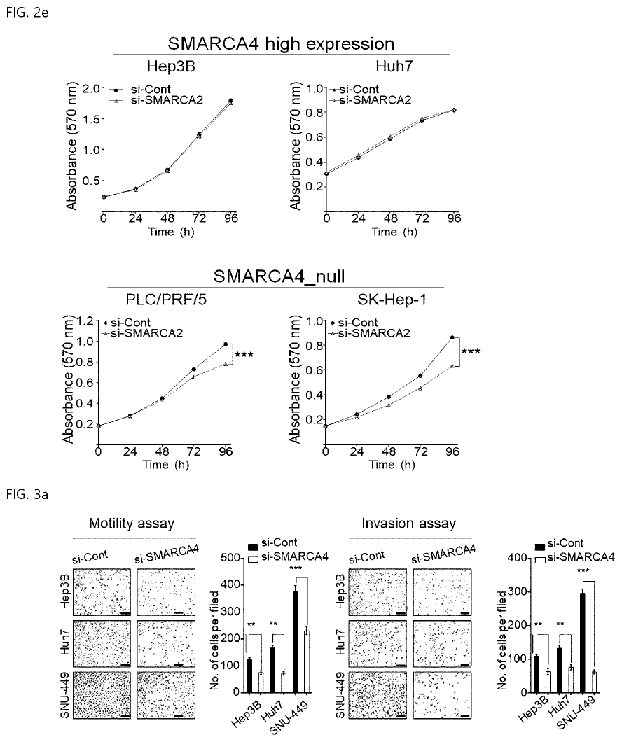

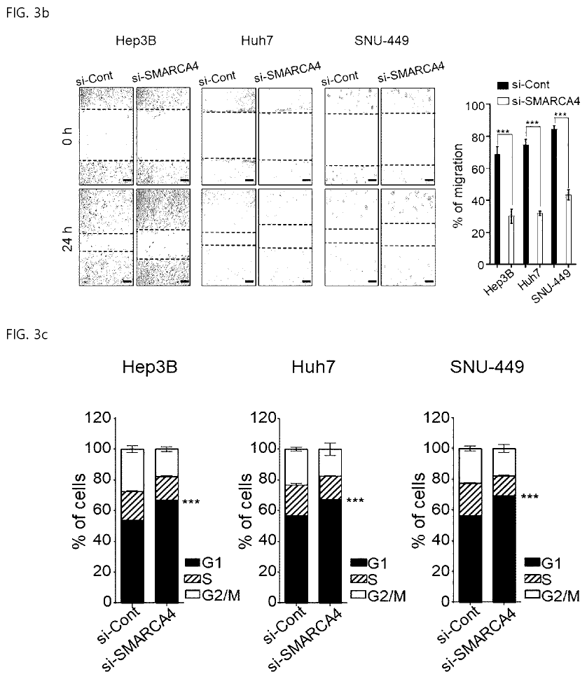

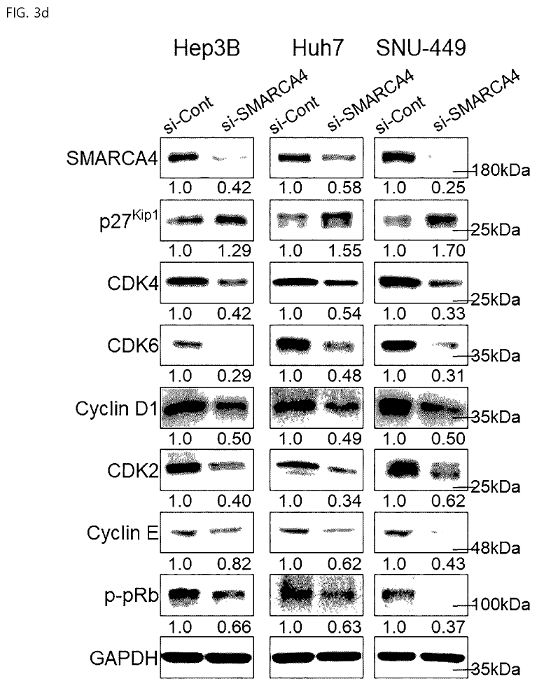

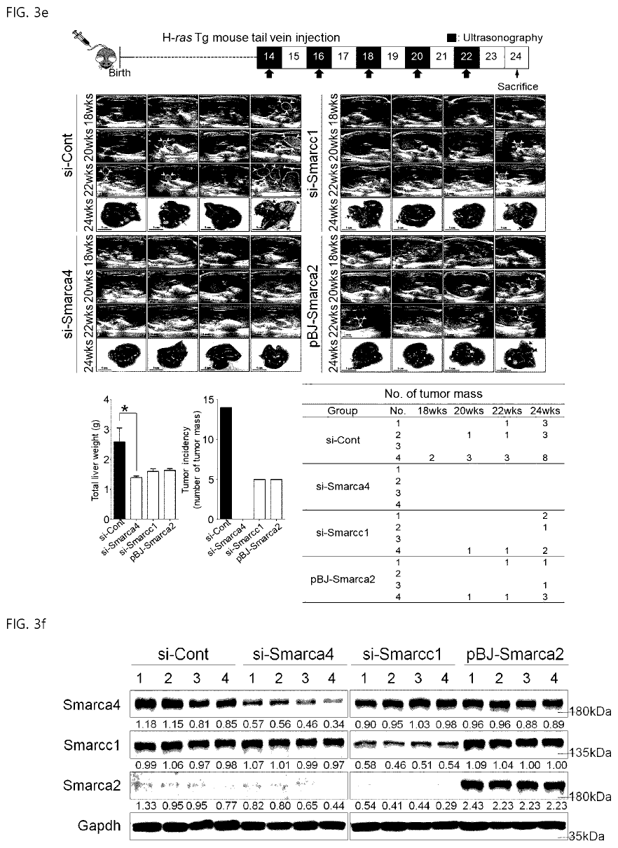

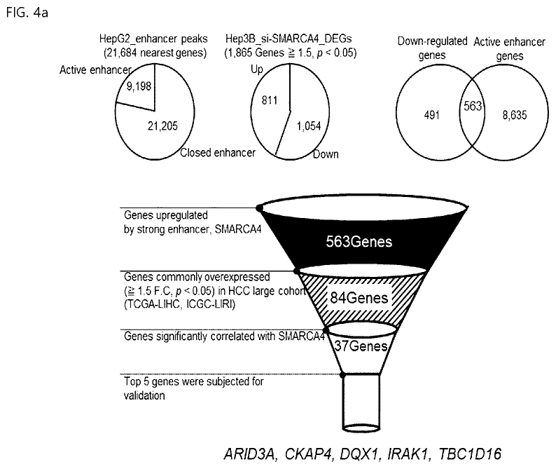

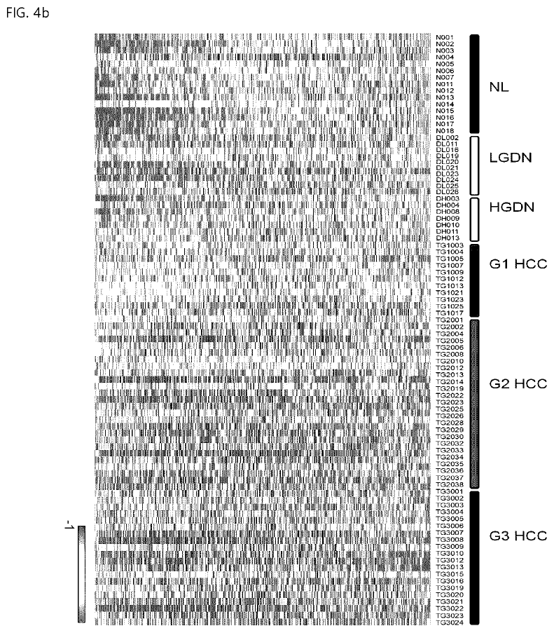

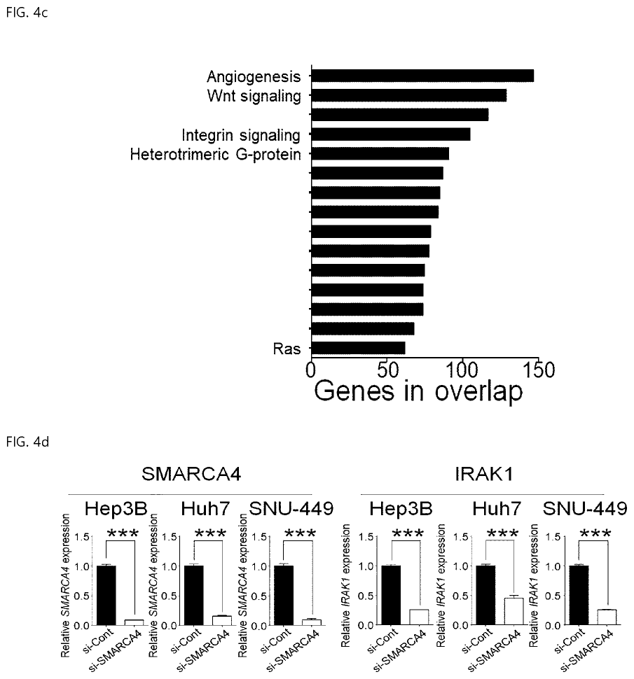

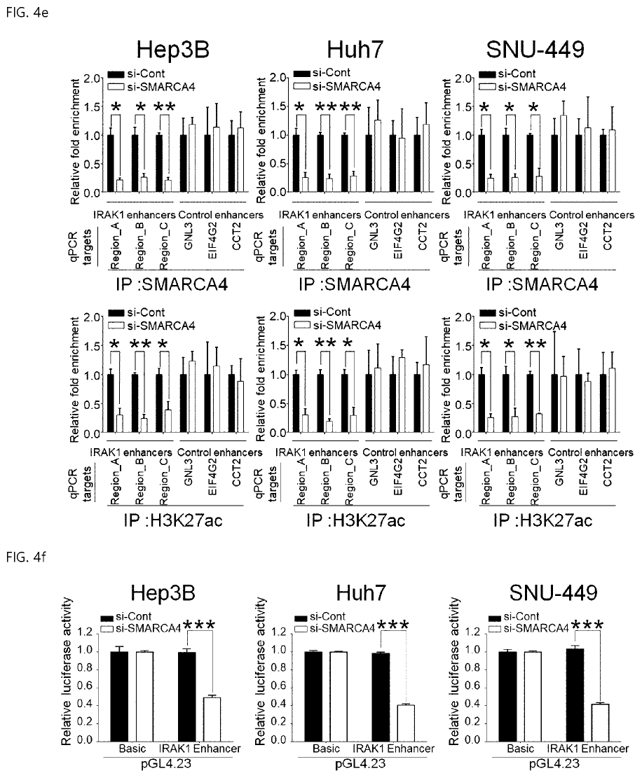

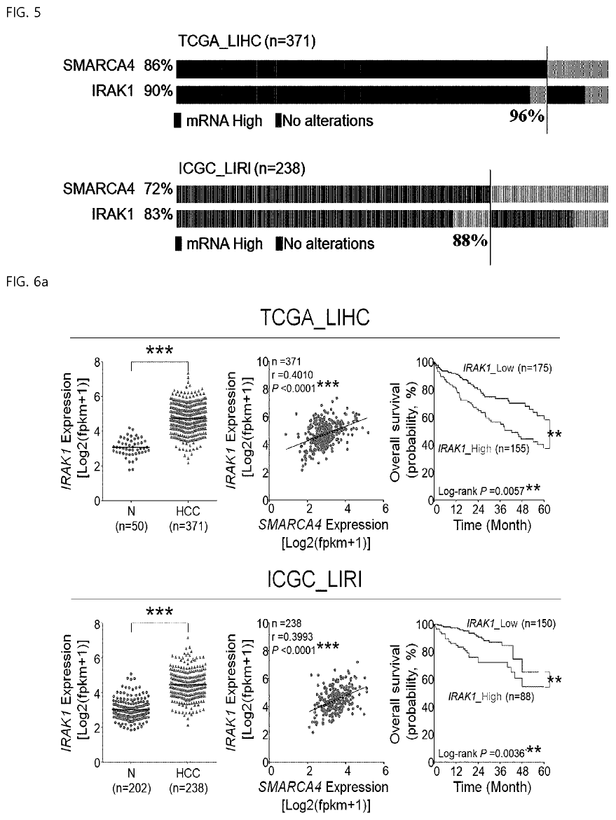

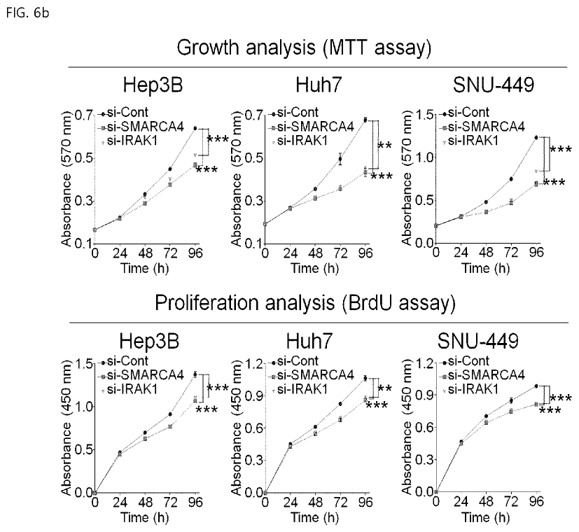

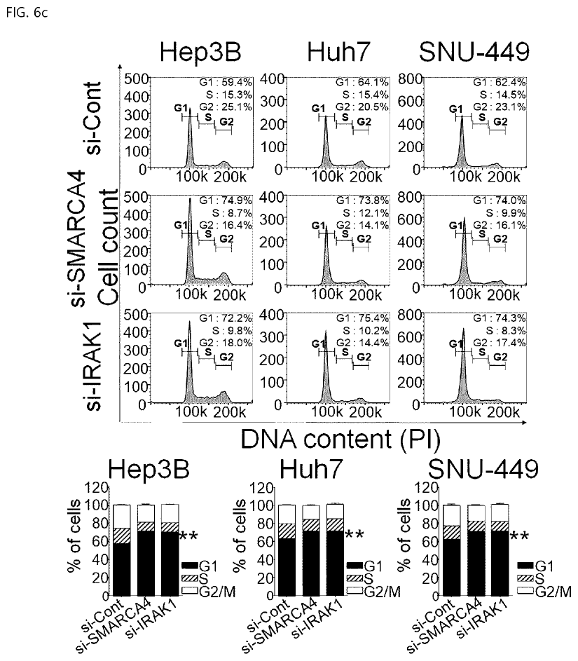

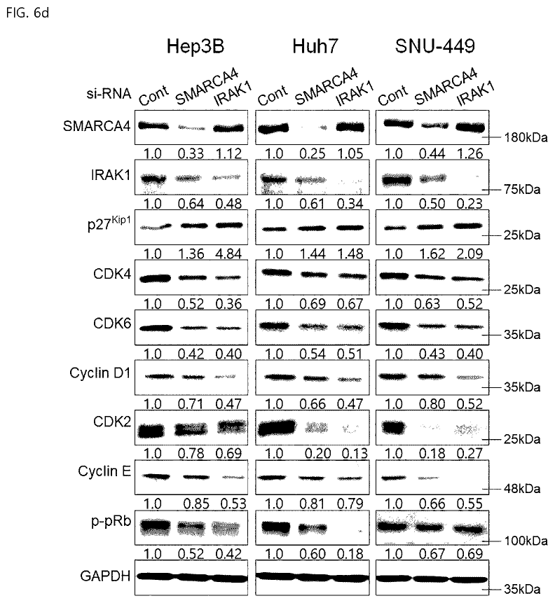

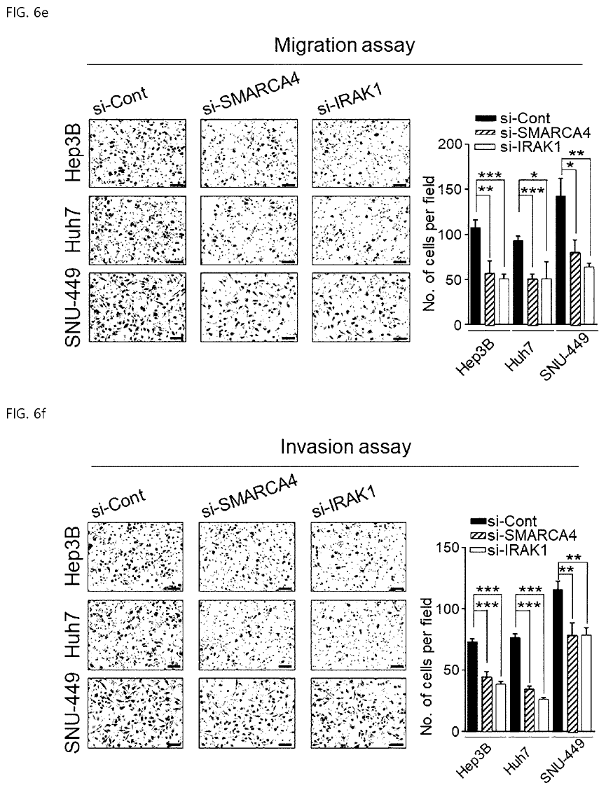

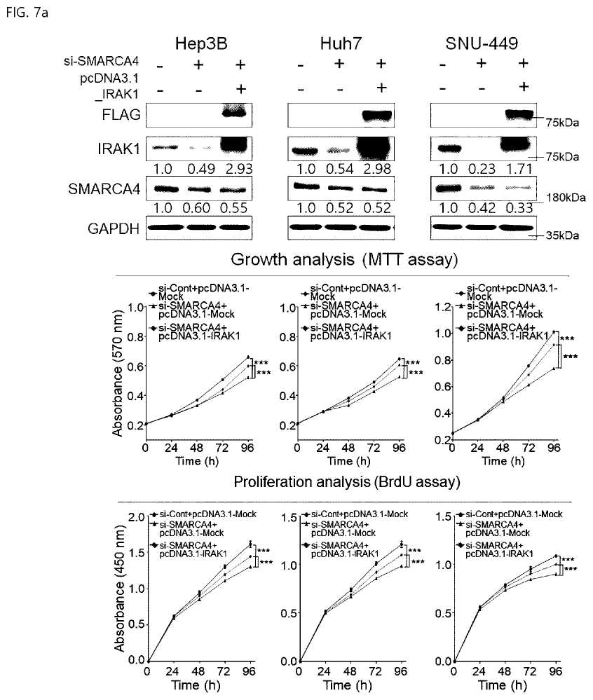

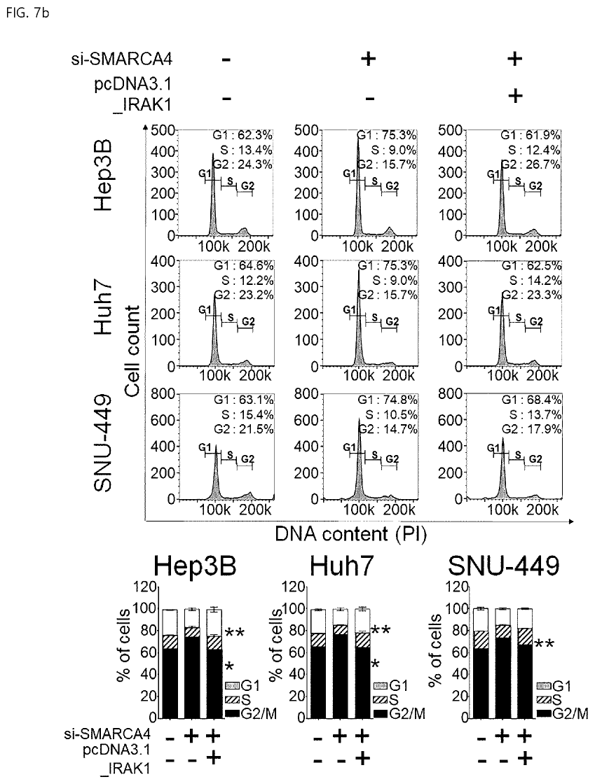

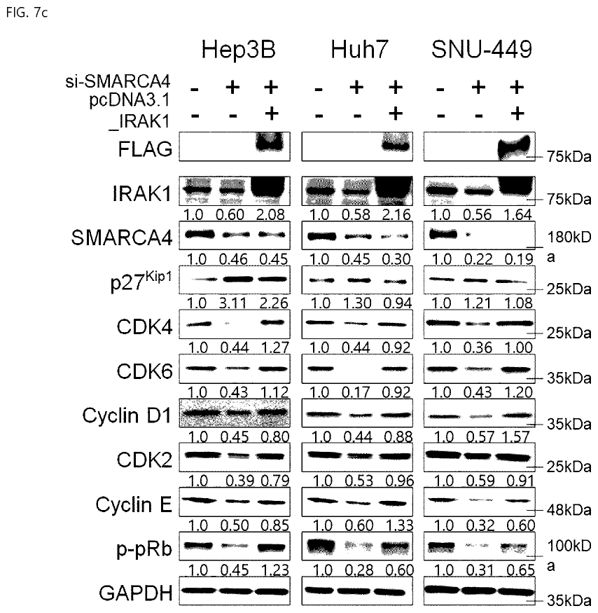

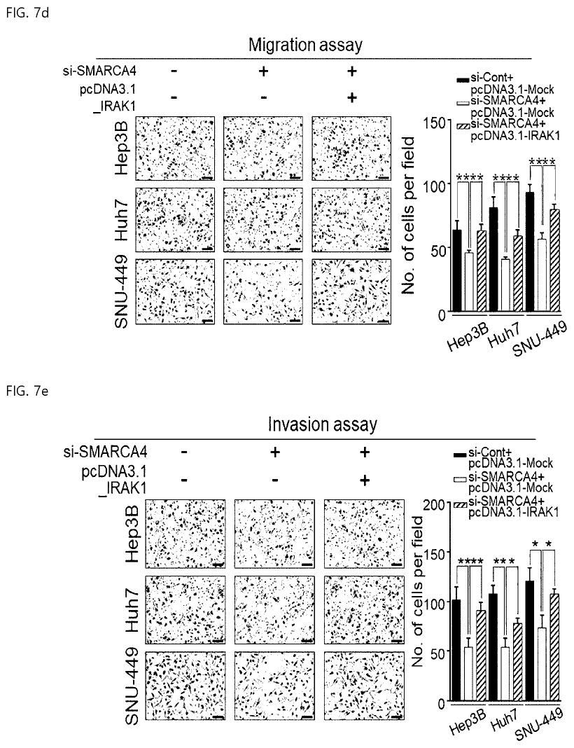

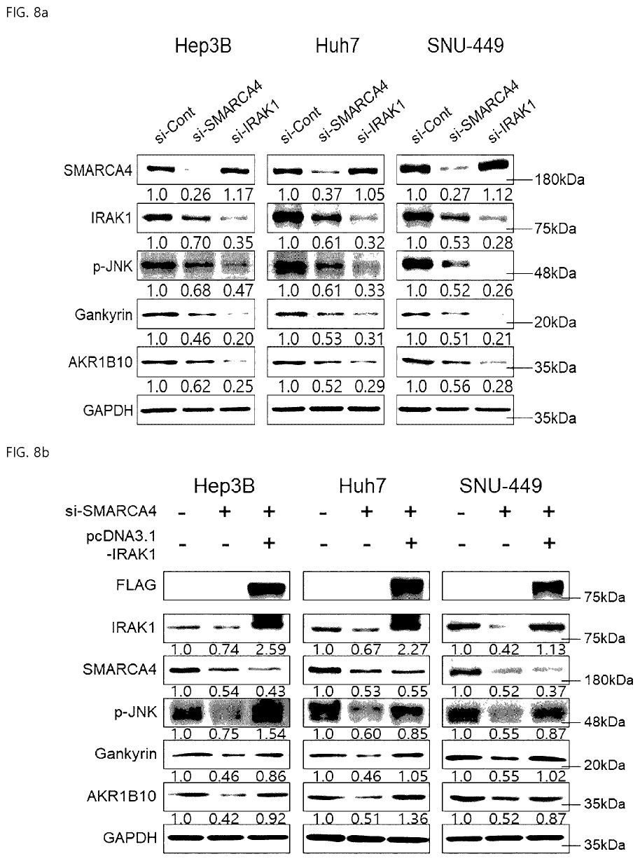

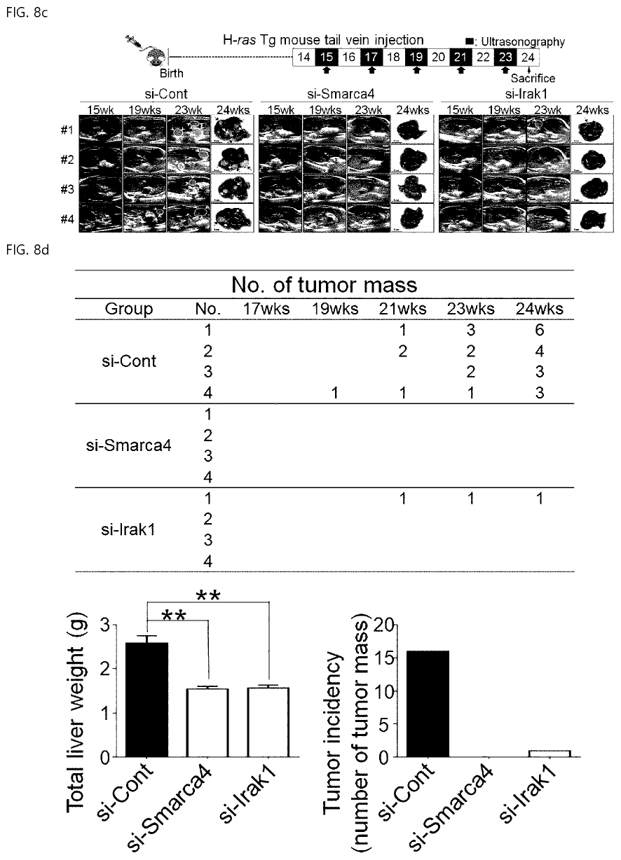

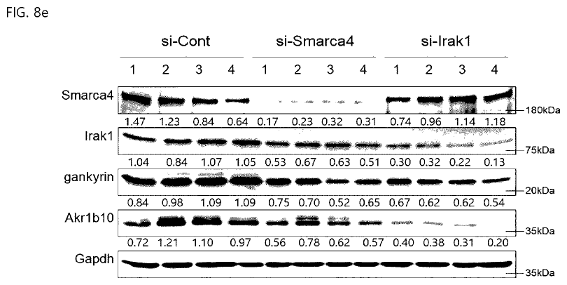

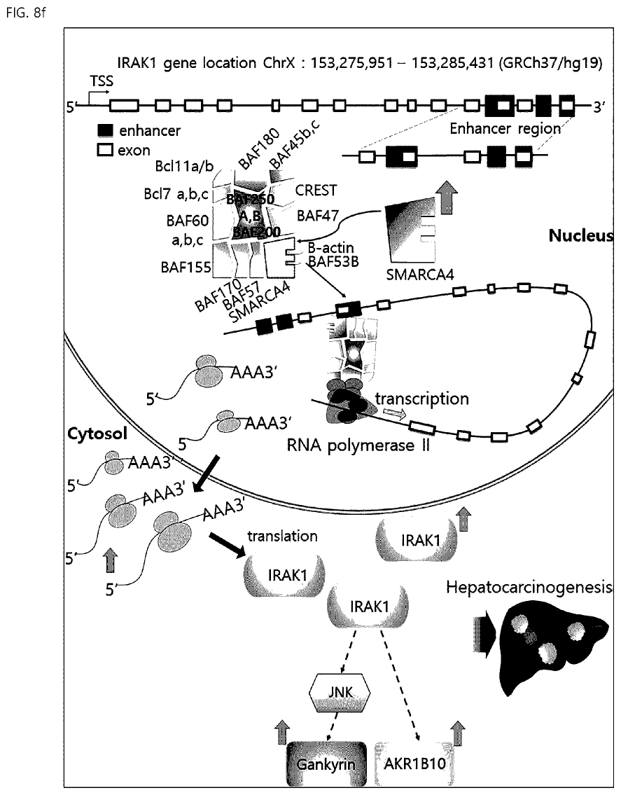

As a result of confirming the overexpression of SMARCA4 and IRAK1 in TCGA_LIHC and ICGC_LIRI datasets, in TCGA_LIHC, among a total of 371 HCC patients, 318 patients (86%) had increased SMARCA4 expression by more than 1.5 times the average SMARCA4 or IRAK1 gene expression value of 50 normal subjects, and 335 patients (90%) had increased IRAK1 expression. Among them, 304 (96%) of 318 patients had both overexpressed SMARCA4 and IRAK1 (FIG. 5). In addition, in ICGC_LIRI, among a total of 238 HCC patients, 172 patients (72%) had increased SMARCA4 expression by more than 1.5 times the average SMARCA4 or IRAK1 gene expression value of 202 normal subjects, and 198 patients (83%) had increased IRAK1 expression, and among them, 152 (88%) of 172 patients had both overexpressed SMARCA4 and IRAK1 (FIG. 5). Through this, it can be inferred that the expression of IRAK1 is increased by SMARCA4.

### Example 5. Confirmation of Tumorigenic Role of IRAK1 in HCC

Since SMARCA4 activated IRAK1 in HCC cells through Examples above, in order to confirm the role of IRAK1 in hepatocellular carcinogenesis, changes in IRAK1 expression were confirmed in TCGA_LIHC and ICGC_LIRI, and as a result, a strong positive correlation between IRAK1 and SMARCA4 was observed (FIG. 6A). In addition, a Kaplan-Meier survival curve of HCC patients showed that the 5-year overall survival rate of patients with high IRAK1 expression was significantly lower than that of patients with low IRAK1 expression (FIG. 6A). Accordingly, in order to confirm the tumorigenic function of IRAK1 in the hepatocellular carcinogenesis, cell growth using MTT assay and cell proliferation using BrdU assay were confirmed in HCC cells. As a result, individual knockdown of SMARCA4 and IRAK1 significantly inhibited tumor cell growth and proliferation in HCC cells (FIG. 6B). In addition, as a result of confirming PI-stained cells by flow cytometry to confirm cell cycle regulation, the knockdown of SMARCA4 and IRAK1 induced cell cycle arrest in HCC cells, respectively. In particular, cell cycle components regulated by SMARCA4 were regulated together with IRAK1, and as a result, it was confirmed that IRAK1 was a downstream molecule regulated by SMARCA4 in HCC (FIGS. 6C and 6D). In addition, the knockdown of SMARCA4 and IRAK1 reduced the migration and invasion responses of chemoattractant-stimulated HCC cells (FIGS. 6E and 6F).

### Example 6. Regulation of IRAK1 Activity by SMARCA4

To confirm whether SMARCA4 directly regulated the IRAK1 activity, HCC cells were knocked down with SMARCA4 siRNA, and a recovery experiment was performed with an IRAK1 expression plasmid (pcDNA3.1_IRAK1). As a result, SMARCA4 knockdown significantly inhibited both tumor cell growth and proliferation of HCC cells, but these anti-tumor effects were lost by co-transformation of the IRAK1 plasmid (FIG. 7A). In addition, as a result of flow cytometry of PI-stained cells, the knockdown of SMARCA4 in HCC cells significantly induced G1-phase arrest, to exhibit an anti-growth effect, whereas the co-transformation of the IRAK1 plasmid into the same cells did not arrest the HCC cell cycle and resumed tumor growth (FIG. 7B). These results were further validated by confirming the cell cycle-related proteins by Western blot assay, and as a result, in HCC, p27 Kip1, which was selectively induced by SMARCA4 knockdown, was inhibited by the IRAK1 plasmid expression, and simultaneously, phosphorylation of cyclin D1, cyclin E, CDK2, CDK4, CDK6, and pRb, of which the expression was suppressed by SMARCA4 knockdown, was restored (FIG. 7C). In addition, chemotaxis-stimulated migration and invasion responses reduced by the SMARCA4 knockdown in HCC cells were also restored by the expression of the IRAK1 plasmid (FIGS. 7D and 7E).

### Example 7. Regulation of Oncoprotein by IRAK1

In order to confirm whether tumor proteins were regulated by SMARCA4-IRAK1, SMARCA4 and IRAK1 were knocked down in HCC cells, and the expression of IRAK1 was restored with an IRAK1 plasmid to confirm the degree of regulation of tumor proteins. As a result, as a result of knocking down both SMARCA4 and IRAK1 in HCC, JNK-dependent Gankyrin and AKR1B10 were significantly inhibited (FIG. 8A). In addition, the inhibitory effect was restored by co-expression of IRAK1 using the IRAK1 plasmid to confirm the regulatory axis of SMARCA4-IRAK1-Gankyrin/AKR1B10 in hepatocellular carcinogenesis (FIG. 8B). In addition, in order to confirm whether the SMARCA4-IRAK1 regulatory axis in vivo may be used as a cancer prevention target, 14-week-old Ras-Tg mice were intravenously injected with Invivofectamine 3.0 (Invitrogen) containing 25 mg/kg of si-SMARCA4 or si-IRAK1, and ultrasonography was performed at 15, 17, 19, 21, and 23 weeks of age (FIG. 8C). As a result, in a control group (si-Cont), HCC was detected at 19 weeks of age, and 4 of 4 mice developed a large number of large tumors. On the other hand, in the si-IRAK1-treated group, HCC was observed at 21 weeks of age, and only relatively small HCC occurred in only 1 of 4 mice, and in the si-SMARCA4-treated group, HCC was not detected at all for 24 weeks. When both SMARCA4 and IRAK1 were targeted, tumor load was reduced, and total liver weight and tumor mass number were also significantly inhibited (FIG. 8D). In addition, even in the Western blot assay, the expression of Gankyrin and AKR1B10 was inhibited in mouse liver in the case of si-SMARCA4 and si-IRAK1-treated groups to demonstrate an in vivo regulatory mechanism through SMARCA4-IRAK1-Gankyrin and AKR1B10 in hepatocellular carcinogenesis (FIGS. 8E and 8F).

1-24. (canceled)

25. A biomarker for diagnosing liver cancer comprising one or more genes selected from the group consisting of SMARCA4 (SWI/SNF related, Matrix associated, actin dependent regulator of chromatin, subfamily a, member 4), SMARCC1, SMARCA2, and IRAK1 (Interleukin-1 receptor-associated kinase 1) or proteins expressed from the genes.

26. The biomarker for diagnosing liver cancer of claim 25, wherein the liver cancer is hepatocellular carcinoma (HCC).

27. The biomarker for diagnosing liver cancer of claim 26, wherein the liver cancer is IRAK1-overexpressed hepatocellular carcinoma.

28. The biomarker for diagnosing liver cancer of claim 25, wherein a gene of Gankyrin or AKR1B10 or a protein expressed from the gene is further included.

29. A composition for diagnosing liver cancer, comprising an agent for measuring an expression level of one or more biomarker genes selected from the group consisting of SMARCA4, SMARCC1, SMARCA2, and IRAK1 at an mRNA or protein level.

30. The composition for diagnosing liver cancer of claim 29, further comprising:
an agent for measuring an expression level of Gankyrin or AKR1B10 at an mRNA or protein level.

31. The composition for diagnosing liver cancer of claim 29, wherein the liver cancer is hepatocellular carcinoma.

32. The composition for diagnosing liver cancer of claim 29, comprising:
an agent for measuring expression levels of SMARCA4 and IRAK1 genes at an mRNA or protein level.

33. The composition for diagnosing liver cancer of claim 29, wherein the agent for measuring the expression level of the biomarker gene at the mRNA level includes a nucleic acid sequence of the marker, a nucleic acid sequence complementary to the nucleic acid sequence, and a primer pair, a probe, or a primer pair and a probe specifically recognizing fragments of the nucleic acid sequence and the complementary sequence.

34. The composition for diagnosing liver cancer of claim 33, wherein the measuring is performed by a method selected from the group consisting of polymerase chain reaction, real-time RT-PCR, reverse transcription polymerase chain reaction, competitive RT-PCR, nuclease protection assay (RNase, 51 nuclease assay), in situ hybridization, nucleic acid microarray, Northern blot, or DNA chip.

35. The composition for diagnosing liver cancer of claim 29, wherein the agent for measuring the expression level of the biomarker gene at the protein level is an antibody, an antibody fragment, an aptamer, an avimer (avidity multimer), or peptidomimetics, that specifically recognizes a full-length protein of the marker or a fragment thereof.

36. The composition for diagnosing liver cancer of claim 35, wherein the measuring is performed by a method selected from the group consisting of Western blot, enzyme linked immunosorbent assay (ELISA), radioimmunoassay (RIA), radioimmunodiffusion, immunoelectrophoresis, tissue immunostaining, immunoprecipitation assay, complement fixation assay, FACS, mass spectrometry, or protein microarray.

37. A kit for diagnosing liver cancer comprising the composition of claim 29.

38. A pharmaceutical composition for preventing or treating liver cancer comprising a SMARCA4 inhibitor as an active ingredient.

39. The pharmaceutical composition for preventing or treating liver cancer of claim 38, further comprising:
SMARCA2 or an expression promoter thereof, a SMARCC1 inhibitor, or an IRAK1 inhibitor.

40. The pharmaceutical composition for preventing or treating liver cancer of claim 38, wherein the liver cancer is liver cancer in which IRAK1 is overexpressed.

41. The pharmaceutical composition for preventing or treating liver cancer of claim 38, wherein the liver cancer is hepatocellular carcinoma.

42. A method for providing information required for diagnosis of liver cancer comprising steps of:
(a) measuring a gene expression level of SMARCA4 or IRAK1 among the biomarker according to claim 25 in a biological sample isolated from a test subject;
(b) comparing a result of the corresponding gene of a normal control sample; and
(c) determining that the test subject has the liver cancer when the gene expression level in step (a) is higher than the expression level of the corresponding gene in step (b).

43. The method for providing information required for the diagnosis of liver cancer of claim 42, further comprising steps of:
(a) measuring a gene or protein expression level of SMARCC1, SMARCA2, Gankyrin, or AKR1B10 in the biological sample isolated from the test subject;
(b) comparing a result of the corresponding gene or protein of the normal control sample; and
(c) determining that the test subject has the liver cancer, when the expression level of SMARCC1, Gankyrin, or AKR1B10 in step (a) is higher than the expression level of the corresponding gene or protein in step (b), or the expression level of SMARCA2 in step (a) is lower than the expression level of the corresponding gene or protein in step (b).

44. A method for preventing or treating liver cancer comprising administering the composition of claim 38 to a subject.
